(19) United States
(12) Patent Application Publication
(10) Pub. No.: US 2012/0109120 A1
(43) Pub. Date: May 3, 2012

(54) Ablation Device

(75) Inventor: Vincent McHugo, Birdhill (IE)

(73) Assignee: Cook Medical Technologies LLC, Bloomington, IN (US)

(21) Appl. No.: 13/278,547

(22) Filed: Oct. 21, 2011

Related U.S. Application Data
(60) Provisional application No. 61/407,644, filed on Oct. 28, 2010.

(51) Int. Cl. A61B 18/18; A61B 18/14

(52) U.S. Cl. 606/33; 606/41

(57) ABSTRACT

An ablation device and a method of ablating a tissue are provided. The ablation device includes a mechanically expandable member having a proximal portion, a distal portion and an energy delivery portion. The mechanically expandable member also has an expanded and a collapsed configuration. The ablation device further includes a first elongate shaft having a proximal portion and a distal portion, the distal portion of the mechanically expandable member is operably connected to the distal portion of the first shaft. The ablation device includes a second elongate shaft having a proximal portion and a distal portion, the proximal portion of the mechanically expandable member is operably connected to the distal portion of the second shaft and the second shaft is movable relative to the first shaft. The ablation device includes a handle operably connected to the first elongate shaft and the second elongate shaft.

### RELATED APPLICATIONS

This application claims the benefit of U.S. Provisional Application No. 61/407,644, filed Oct. 28, 2010, which is incorporated by reference herein in its entirety.

### BACKGROUND

Millions of people suffer from progressive gastroesophageal reflux disease (GERD) which is characterized by frequent episodes of heartburn, typically on at least a daily basis. Without adequate treatment, GERD can cause erosion of the esophageal lining as the lower esophageal sphincter (LES), a segment of smooth muscle located at the junction of the stomach and the esophagus, gradually loses its ability to function as the barrier that prevents stomach acid reflux. Chronic GERD can also cause metaplasia to the inner lining of the esophagus where the normal squamous mucosa changes to columnar mucosa, also known as Barrett's esophagus. Barrett's esophagus can progress to esophageal cancer if left untreated.

Endoscopic treatment of Barrett's esophagus includes endoscopic mucosal resection (EMR). One method of performing EMR involves ablation of the mucosal surface by heating the surface until the surface layer is no longer viable. The dead tissue is then removed.

Treatment devices for performing EMR have been developed using bipolar ablation technology that includes circumferentially oriented electrodes to endoscopically ablate the diseased tissue. Typically, the circumferentially oriented electrodes are positioned on an inflatable balloon. The balloon must be inflated to a predetermined size to achieve adequate contact with the diseased tissue for delivery of the appropriate amount of energy from the bipolar ablation device to ablate the diseased tissue. In order to determine the correct size and balloon pressure to achieve adequate ablation, a sizing balloon must first be introduced into the esophagus. Once the proper measurements are made with the sizing balloon, the treatment device can then be endoscopically inserted into the patient's esophagus. The balloon-inflated treatment device and procedure requires an additional step to size the balloon and adds more time and potential patient discomfort to the treatment procedure. In addition, the inflated balloon is positioned in front of the endoscope viewing window, preventing direct visualization of the target tissue and potentially leading to ablation of healthy tissue or incomplete ablation of diseased tissue. Balloon inflation also relies on the movement of gas or liquid to move the balloon from the delivery position collapsed against the catheter to the inflated position. The time required for inflation of the balloon increases the amount of time required for the procedure.

What is needed in the art is an ablation treatment device that is simple to use and that minimizes the number of steps in a treatment procedure. A rapidly expandable and collapsible device is also desirable.

### BRIEF SUMMARY

Accordingly, it is an object of the present invention to provide a device and a method having features that resolve or improve on one or more of the above-described drawbacks.

One embodiment of the ablation device includes a mechanically expandable member having a proximal portion, a distal portion and an energy delivery portion. The mechanically expandable member also has an expanded configuration and a collapsed configuration. The ablation device further includes a first elongate shaft having a proximal portion and a distal portion, the distal portion of the mechanically expandable member is operably connected to the distal portion of the first shaft. The ablation device includes a second elongate shaft having a proximal portion and a distal portion, the proximal portion of the mechanically expandable member is operably connected to the distal portion of the second shaft and the second shaft movable relative to the first shaft. The ablation device includes a handle operably connected to the first elongate shaft and the second elongate shaft where movement of the handle changes a position of the first shaft relative to the second shaft to move the mechanically expandable member from the collapsed configuration to the expanded configuration.

In another embodiment, a method of ablating a tissue is provided. The method includes inserting a distal portion of an ablation device into a lumen of a patient. The ablation device includes a mechanically expandable member having a proximal portion, a distal portion and an energy delivery portion. The ablation device also includes a first elongate shaft having a proximal portion and a distal portion. The distal portion of the mechanically expandable member is operably connected to the distal portion of the first shaft. The ablation device includes a second elongate shaft having a proximal portion and a distal portion. The proximal portion of the mechanically expandable member is operably connected to the distal portion of the second shaft. The second shaft is movable relative to the first shaft. A handle operably connected to the first elongate shaft and the second elongate shaft. The method further includes positioning a portion of the mechanically expandable ablation member at a treatment site, moving the first shaft in a first direction relative to the second shaft to move the mechanically expandable ablation member from the collapsed configuration to the expanded configuration and applying energy to the tissue from the energy source.

### BRIEF DESCRIPTION OF THE DRAWINGS

FIG. 1 is a sectional view of a mechanically expandable ablation device in accordance with an embodiment of the present invention;

FIG. 2 is sectional view of the ablation device shown in FIG. 1 in a collapsed configuration;

FIG. 3 is a cross-sectional view of the ablation device shown in FIG. 1;

FIG. 4 is a partial view of an inner layer of the ablation device;

FIG. 5 is a partial view of an outer layer of the ablation device;

FIGS. 6A-6C illustrate exemplary patterns for electrodes of the ablation device;

FIG. 7 is a partial view of an embodiment of a proximal portion of the ablation device;

FIG. 8 is a partial sectional view an embodiment of the distal portion of the ablation device; and

FIGS. 9A-9C illustrate operation of the ablation device.

### DETAILED DESCRIPTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the ablation device to a patient. Hence the term “distal” means the portion of the ablation device that is farthest from the physician and the term “proximal” means the portion of the ablation device that is nearest to the physician.

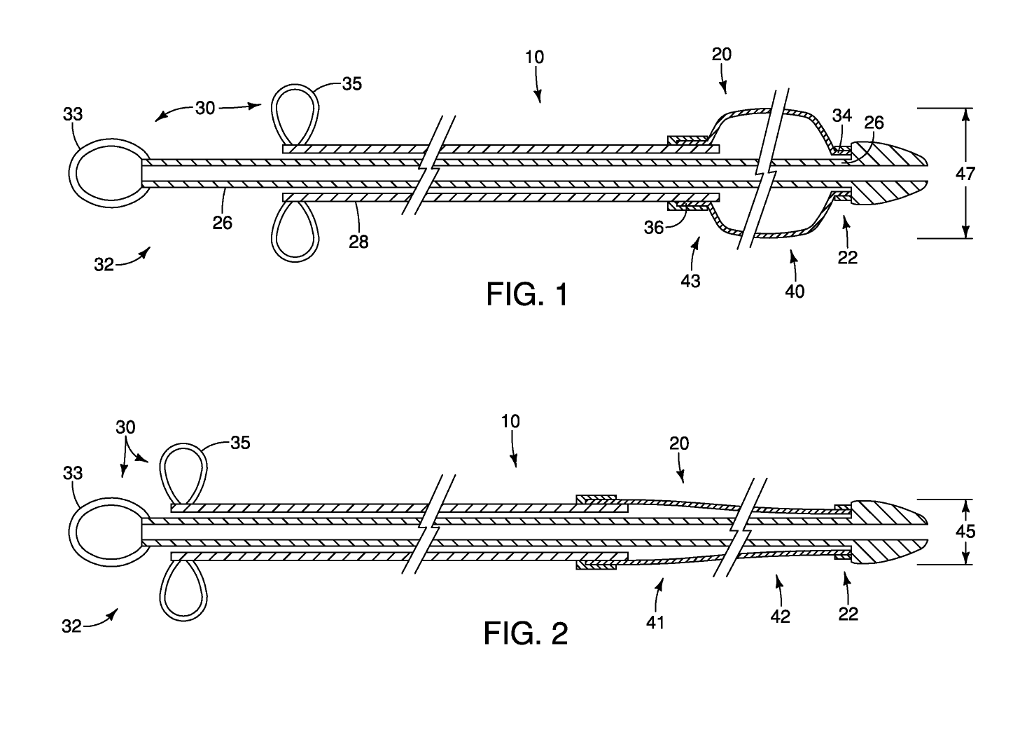

FIG. 1 illustrates an embodiment of an ablation device 10 in accordance with the present invention. The ablation device 10 includes a mechanically expandable ablation member 20 at a distal portion 22 of the device 10. The mechanically expandable ablation member 20 is operably connected to an inner shaft 26 and an outer shaft 28. In some embodiments, the inner shaft 26 is coaxially positioned within the outer shaft 28 as shown in FIG. 1. The mechanically expandable member 20 expands and collapses by longitudinal movement of the inner shaft 26 relative to the outer shaft 28 as explained in more detail below and not by inflation and deflation of a balloon. The term “mechanically expandable” as used herein refers to a device that is expandable by other than air or fluid expansion. A control handle 30 is provided at a proximal portion 32 of the device 10. The handle 30 is operable to control the movement of the inner and outer shafts 26, 28 relative to each other. The handle 30 may be any type of handle that is operable to control the movement of the inner shaft 26 relative to the outer shaft 28.

As shown in FIG. 1, a distal portion 34 of the mechanically expandable member 20 is operably connected to the inner shaft 26. A proximal portion 36 of the mechanically expandable member 20 is operably connected to the outer shaft 28. Relative movement of the inner and outer shafts 26, 28 causes the expandable member 20 to move between an expanded configuration 40 shown in FIG. 1 and a collapsed configuration 42 shown in FIG. 2. By way of non-limiting example, the relative movement of the inner and the outer shafts 26, 28 may be longitudinal movement or rotational movement. The mechanically expandable member 20 in the collapsed configuration 42 has a first diameter 45 and the mechanically expandable member 20 in the expanded configuration 40 has a second diameter 47. The second diameter 47 is greater than the first diameter 45. The collapsed configuration 42 may be used to deliver the ablation device 10 to a treatment site within the patient and for repositioning the ablation device 10 within the patient's lumen to provide treatment to additional sites if needed. In some embodiments, the distal portion 34 of the mechanically expandable member 20 may be connected to the outer shaft 28 and the proximal portion 36 of the mechanically expandable member 20 may be connected to the inner shaft 26.

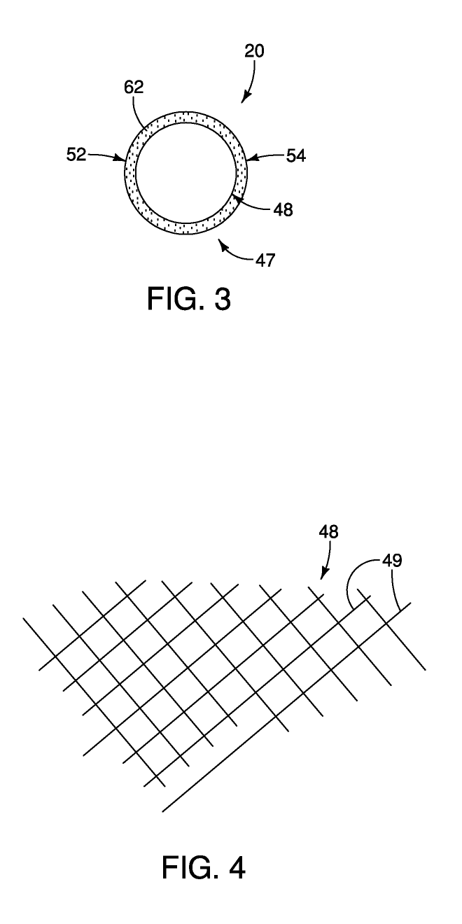

A cross-sectional view of an embodiment of the mechanically expandable member 20 is shown in FIG. 3. The mechanically expandable member 20 may include one or more layers 47. As shown in FIG. 3, the expandable member 20 may include an inner layer 48 connected to the inner and outer shafts 26, 28, an intermediate layer 52 and an outer layer 54. The intermediate layer 52 may be an insulating layer and the outer layer 54 may include one or more electrodes configured to contact tissue at a treatment site.

The inner layer 48 may be formed from a material that is expandable and collapsible in response to movement of the inner and outer shafts 26, 28 relative to each other. The inner layer 48 has sufficient strength and/or rigidity to support additional layers 47 and to position the outer layer 54 against the tissue at the treatment site. An exemplary expandable material for the inner layer 48 is shown in FIG. 4 as a mesh having a plurality of interwoven wire or polymer filaments 49 or combinations of wire and polymer filaments 49. In some embodiments, by way of non-limiting example, the mesh may be formed from woven double helical wire. The mesh may be formed from wire such as nickel titanium alloys, for example, nitinol, stainless steel, cobalt alloys and titanium alloys. In some embodiments, the mesh may be formed from a polymeric material such as a polyolefin, a fluoropolymer, a polyester, for example, polypropylene, polytetrafluoroethylene, polyvinylidene fluoride, polyethylene terephthalate (PET), and combinations thereof. Other materials known to one skilled in the art may also be used to form the inner layer 48 provided that the material is moveable between the expanded and collapsed configurations 40, 42 in response to the inner shaft 26 moving relative to the outer shaft 28. In some embodiments, the inner layer 48 may be a framework that is expandable. By way of non-limiting example, the framework may include longitudinally oriented ribs, a coil or a self expanding stent like structure. In some embodiments, the inner layer 48 may be supported on the framework.

The intermediate layer 52 may optionally be included when the inner layer 48 is made from an electrically conductive material. The intermediate layer 52 may be an insulating layer to provide an insulating barrier between the outer layer 54 and the inner layer 48. In some embodiments, the intermediate layer 52 may be a coating 62 that is applied to the inner layer 48 in a quantity that is sufficient to insulate the inner layer 48 from the outer layer 54. In some embodiments, the coating 82 may be made from parylene-N (poly-p-xylylene). Other xylylene polymers, and particularly parylene polymers, may also be used as a coating within the scope of the present invention, including, for example, 2-chloro-p-xylylene (Parylene C), 2, 4-dichloro-p-xylylene (Parylene D), poly(tetraflouro-p-xylylene), poly(carboxyl-p-xylylene-co-p-xylylene), fluorinated parylene, or parylene HT® (a copolymer of per-fluorinated parylene and non-fluorinated parylene), alone or in any combination. Preferred coatings of the present will include the following properties: low coefficient of friction (preferably below about 0.5, more preferably below about 0.4, and most preferably below about 0.35); very low permeability to moisture and gases; fungal and bacterial resistance; high tensile and yield strength; high conformality (ready application in uniform thickness on all surfaces, including irregular surfaces, without leaving voids); radiation resistance (no adverse reaction under fluoroscopy); bio-compatible/bio-inert; acid and base resistant (little or no damage by acidic or caustic fluids); ability to be applied by chemical vapor deposition bonding/integrating to wire surface (bonding is intended to contrast to, for example, fluoroethylenes that form surface films that are able to be peeled off an underlying wire); and high dielectric strength. The intermediate layer 52 may also be provided as a separate layer 47 that is movable with the inner layer 48 as the inner layer 48 is moved between the expanded and collapsed configurations 40, 42 and provides insulation between the inner layer 48 and the outer layer 54. For example, the intermediate layer 52 may be provided as an elastomeric layer formed of a polymer, such as polyethylene terephthalate (PET), polyimide, polyamide, silicone, latex or rubber. Additional materials known to one skilled in the art may also be used as the intermediate layer 52.

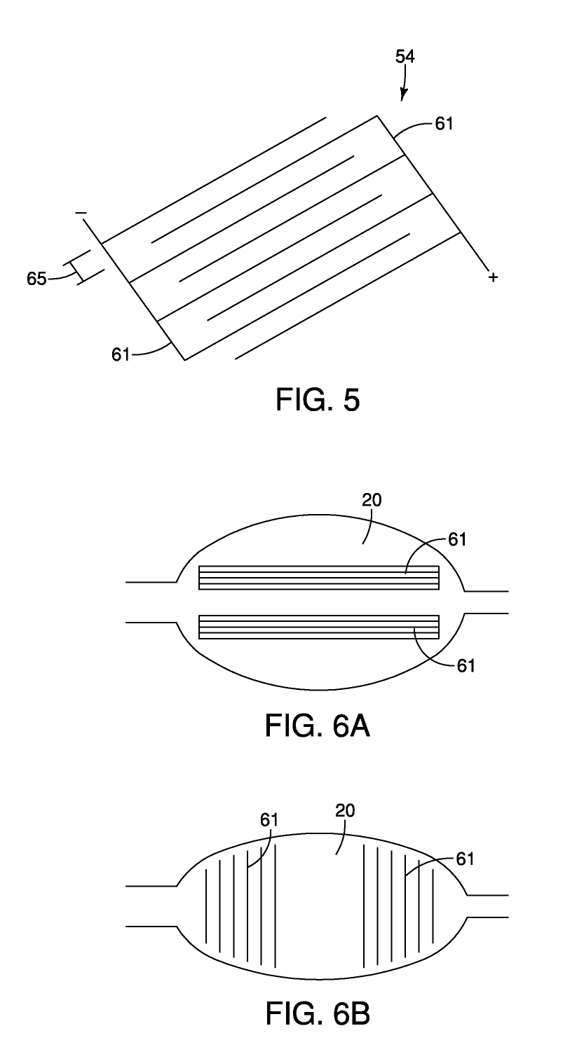

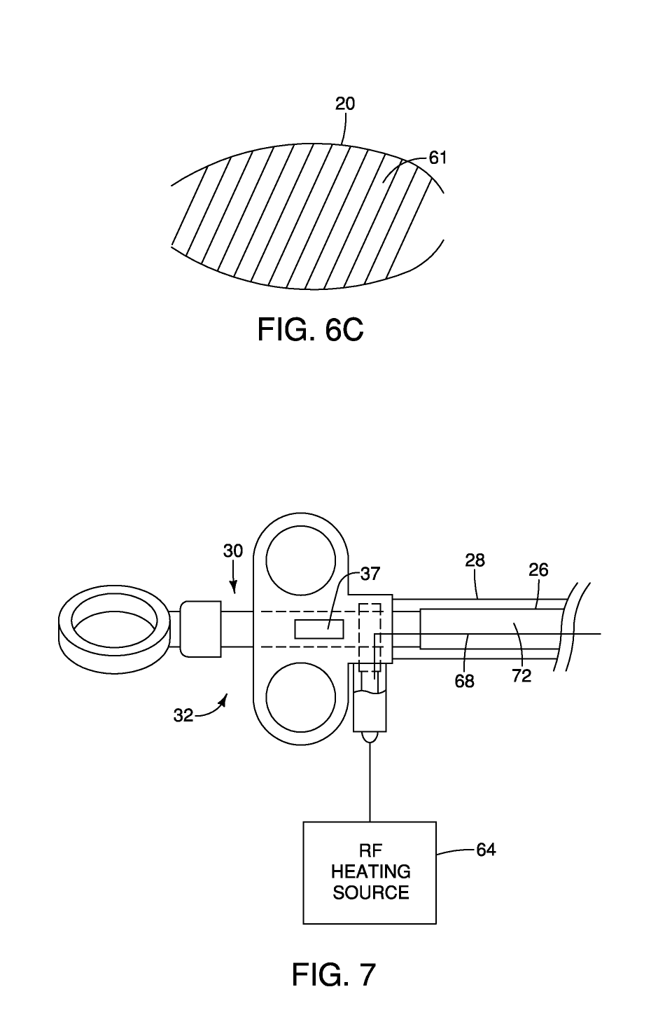

FIG. 5 illustrates an exemplary outer layer 54 including a plurality of electrodes 61. As shown, the outer layer 54 includes a positive electrode 61 and a negative electrode 61 in a bipolar device. The outer layer 54 may be provided with a plurality of electrodes 61 and when provided as a bipolar device the electrodes 61 are provided in pairs, one positive and one negative electrode per pair. The outer layer 54 may also be provided as a monopolar device having a single electrode 61 or a plurality of electrodes 61 with a grounding pad or an impedance circuit additionally provided (not shown). The electrodes 61 may be provided in any pattern on the mechanically expandable member 20. The electrodes 61 may cover the entire expandable ablation member 20 or a portion of the expandable ablation member 20. By way of non-limiting example, the electrodes 61 may be provided in a longitudinal pattern covering all or a portion of the expandable member 20 as shown in FIG. 6A. The electrodes 61 may be provided in a radial pattern covering all or a portion of the mechanically expandable member 20 as shown in FIG. 6B. In some embodiments, the electrodes 61 may be provided in an angular pattern or a helical pattern covering all or a portion of the expandable member 20 as shown in FIG. 6C. Space 65 between the electrodes 61 may be optimized to control the depth of ablation of the target tissue. By way of non-limiting example, the space 65 between the positive electrode portion 61 and the negative electrode portion 61 may between about 0.1 mm to about 5 mm. Other spacing distances between electrodes are also possible and depend on the target tissue, the depth of the lesion, the type of energy and the length of application of the energy to the tissue.

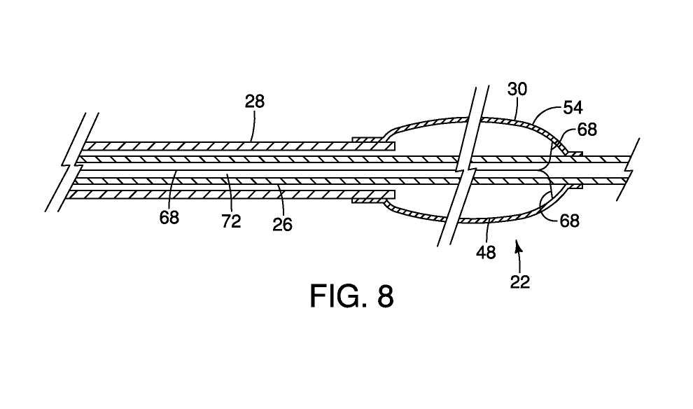

The electrodes 61 are operably connected to an energy source 64. As shown in FIG. 7, the handle 30 may include a connector 66 for operably connecting the electrodes 61 to the energy source 64. As shown, the energy source 64 may be a radio frequency source. However, other types of energy sources 64 may also be used to provide energy to the mechanically expandable member 20. By way of non-limiting example, additional possible energy sources may include microwave, ultraviolet, cryogenic and laser energies. The electrodes 61 may be connected to the power source 64 by an electrical conductor, such as one or more wires 68 that extend from the electrodes 61 to the connector 66 that connects to the energy source 64. The wires 68 may extend through a lumen 72 of the inner shaft 26 as shown in FIG. 8. Alternatively, the wires 68 may extend through a lumen of the outer shaft 28 or external to the outer shaft 28 and may optionally include a sleeve surrounding the shaft 28 and the wires 68 (not shown).

As discussed above, the handle 30 is operable to move the inner shaft 26 relative to the outer shaft 28 so that the mechanically expandable member 20 moves between the expanded configuration 40 and the collapsed configuration 42 (see FIGS. 1 and 2). By way of non-limiting example, the handle 30 includes a first portion 33 and a second portion 35 that move relative to each other. As shown in FIG. 1, the first portion 33 is operably connected to the inner shaft 26. The second portion 35 is operably connected to the outer shaft 28. The first portion 33 may be moved proximally and/or the second portion 35 may be moved distally to move the inner shaft 26 proximally and/or the outer shaft 28 distally to move the expandable member 20 to the expanded configuration 40 as shown in FIG. 1. As shown in FIG. 2, the first portion 33 may be moved distally and/or the second portion moved proximally to move the inner shaft 26 distally and/or the outer shaft 28 proximally to move the expandable member 20 to the collapsed configuration 42. Movement of the inner shaft 26 relative to the outer shaft 28 moves the distal portion of 34 of the expandable member 20 proximally relative to the distal portion 36 of the mechanically expandable member 20 so that the diameter of the expandable member 20 increases relative to the collapsed configuration 42.

The handle 30 may include a lock 37 shown in FIG. 7 to releasably lock the first portion 33 in position relative to the second portion and thus lock the expandable member 20 in position. The lock 37 may releasably lock the first and second portions 33, 35 of the handle 30 together at any proximal/distal positioning of the inner and outer shafts 26, 28 so that the expandable member 20 may be locked at any size that is suitable for the treatment site. For example, if the treatment site is in a narrow lumen, the first portion 33 of the handle 30 may be moved slightly in the proximal direction to give the expandable member 20 a smaller diameter than if the first portion 33 were moved fully distally to give the expandable member 20 the largest diameter.

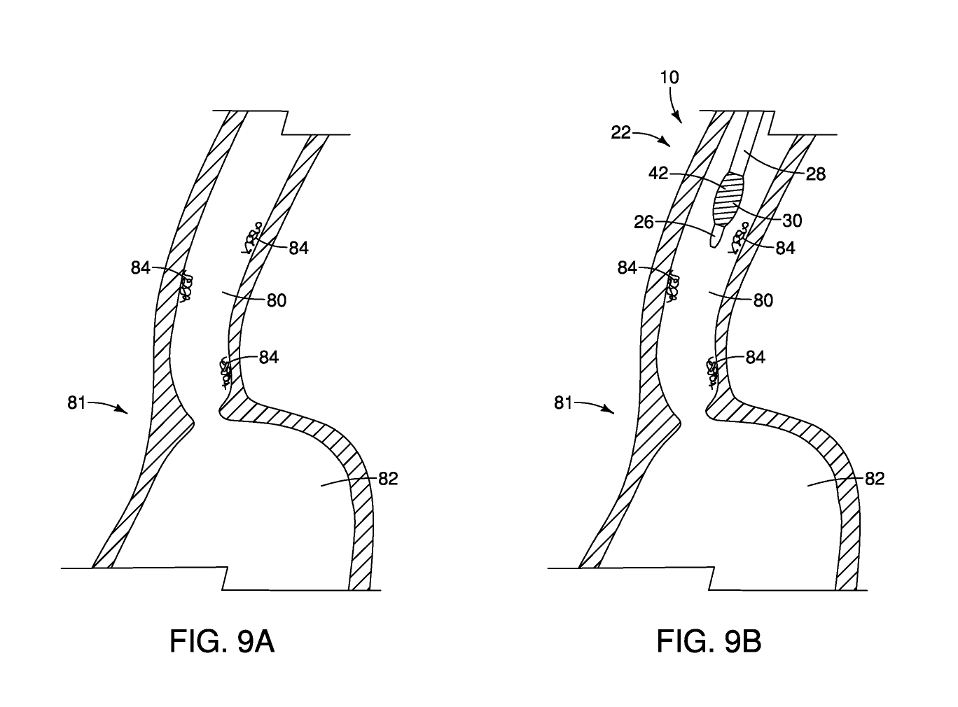

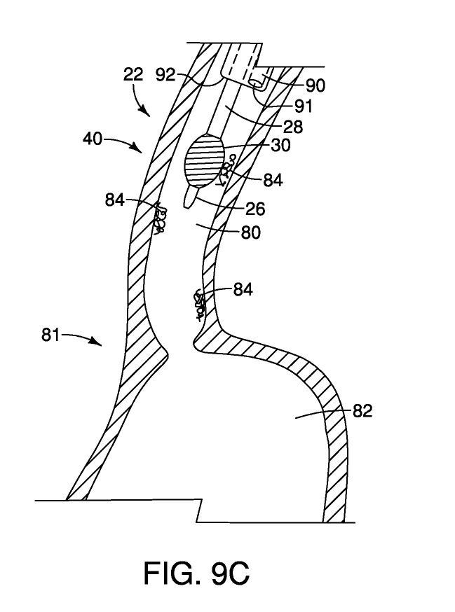

Operation of the ablation device 10 will be explained with reference to FIGS. 9A-9C. FIG. 9A illustrates a patient's esophagus 80, lower esophageal sphincter (LES) 81 and stomach 82. Areas of diseased tissue 84 within the esophagus 80 are also shown. The diseased tissue 84 may be columnar mucosa (Barrett's esophagus) that is to be ablated using the ablation device 10. FIG. 9B illustrates the distal portion 22 of the ablation device 10 being inserted into the patient's esophagus 80. The ablation device 10 is inserted into the esophagus 80 with the mechanically expandable member 20 in the collapsed configuration 42 for delivery to the proper position. The inner shaft 26 is in a first position 41 relative to the outer shaft 28 (also shown in FIG. 2). In some situations, the ablation device 10 may be delivered using an endoscope 90 that is shown in FIG. 9C to facilitate placement of the expandable member 20 in the proper position to ablate the diseased tissue 84. The endoscope 90 may include a viewing port 91 for visualizing the diseased tissue 84 and positioning the ablation device 10. The endoscope 90 may also include a working channel 92 and a flush port. The ablation device 10 may be delivered through the working channel 92 or optionally back-loaded into the working channel 92 before insertion of the endoscope 90 into the patient. As shown in FIG. 9C, the distal portion 22 of the ablation device ablation device 10 is delivered through the working channel 92 and positioned so that the mechanically expandable member 20 is adjacent to the diseased tissue 84 in the expanded configuration 40. The inner shaft 26 is in a second position 41 relative to the outer shaft 28 (also shown in FIG. 1). The outer layer 54 of the expandable ablation member 20 may be positioned so that the outer layer 54 directly contacts the diseased tissue 84 or an electroconductive fluid may be provided between the outer layer 54 and the diseased tissue 84. The power source 64 is activated for a sufficient time to ablate the diseased tissue 84. The expandable member 20 may then be collapsed to the collapsed configuration 42 by moving the inner shaft 26 relative to the outer shaft 28 to return to the first position 41. The expandable member 20 may be repositioned at a new tissue site or removed once the ablation of the diseased tissue is completed. While the procedure has been described with reference to the ablation of diseased tissue in the esophagus using the ablation device 10, the location of the treatment is not limited to the esophagus. By way of non-limiting example, portions of the stomach, the gastrointestinal tract, the lungs or the vascular system may also be treated using the ablation device 10. For example, the device 10 may be used for treating bleeding varices in the esophagus or for treatment of prostatic diseases, such as benign prostatic hyperplasia.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. An ablation device comprising:
a mechanically expandable member comprising a proximal portion and a distal portion, the mechanically expandable member having an expanded configuration and a collapsed configuration; the mechanically expandable member comprising an energy delivery portion;
a first elongate shaft having a proximal portion and a distal portion, the distal portion of the mechanically expandable member operably connected to the distal portion of the first shaft;
a second elongate shaft having a proximal portion and a distal portion; the proximal portion of the mechanically expandable member operably connected to the distal portion of the second shaft; the second shaft movable relative to the first shaft; and
a handle operably connected to the first elongate shaft and the second elongate shaft;
wherein movement of the handle changes a position of the first shaft relative to the second shaft to move the mechanically expandable member from the collapsed configuration to the expanded configuration.

2. The ablation device of claim 1, wherein the energy delivery portion comprises an electrode.

3. The ablation device of claim 1, wherein the mechanically expandable member comprises a plurality of layers.

4. The ablation device of claim 3, wherein the mechanically expandable member comprises an inner layer that is operably connected to the first shaft and the second shaft and an outer layer comprising the energy delivery portion overlaying at least a portion of the inner layer.

5. The ablation device of claim 4, wherein the mechanically expandable member comprises an intermediate layer providing insulation between the inner layer and the outer layer.

6. The ablation device of claim 5, wherein the intermediate layer comprises a coating.

7. The ablation device of claim 1, wherein the mechanically expandable member comprises a mesh.

8. The ablation device of claim 7, wherein the mesh comprises a wire or a polymer.

9. The ablation device of claim 1, wherein the first shaft is longitudinally movable relative to the second shaft.

10. The ablation device of claim 1, wherein the handle further comprises a connector for operably connecting the energy delivery portion to a power source.

11. The ablation device of claim 10, wherein the power source delivers radio frequency energy.

12. The ablation device of claim 1, wherein the first shaft and the second shaft are coaxially positioned and longitudinally movable relative to each other.

13. The ablation device of claim 1, wherein the ablation device is a bipolar device.

14. The ablation device of claim 1, further comprising an endoscope having a working channel, the ablation device deliverable to a treatment site using the working channel.

15. An ablation device comprising:
a mechanically expandable member comprising a proximal portion and a distal portion, the mechanically expandable member having an expanded configuration and a collapsed configuration; the mechanically expandable member comprising an energy delivery portion;
a first elongate shaft having a proximal portion and a distal portion, the distal portion of the mechanically expandable member operably connected to the distal portion of the first shaft; and
a second elongate shaft having a proximal portion and a distal portion; the proximal portion of the mechanically expandable member operably connected to the distal portion of the second shaft;
wherein movement of the first shaft relative to the second shaft moves the mechanically expandable member from the collapsed configuration to the expanded configuration.

16. A method of ablating a tissue, the method comprising:
inserting a distal portion of an ablation device into a lumen of a patient, the ablation device comprising:
a mechanically expandable member comprising a proximal portion and a distal portion, the mechanically expandable member having an expanded configuration and a collapsed configuration; the mechanically expandable member comprising an energy delivery portion;
a first elongate shaft having a proximal portion and a distal portion, the distal portion of the expandable member operably connected to the distal portion of the first shaft;
a second elongate shaft having a proximal portion and a distal portion; the proximal portion of the mechanically expandable member operably connected to the distal portion of the second shaft; the second shaft movable relative to the first shaft; and
a handle operably connected to the first elongate shaft and the second elongate shaft;
positioning a portion of the expandable ablation member at a treatment site;
moving the first shaft in a first direction relative to the second shaft to move the mechanically expandable member from the collapsed configuration to the expanded configuration; and
applying energy to the tissue from the energy source.

17. The method of claim 16, comprising longitudinally moving the first shaft in a second direction substantially opposite to the first direction to collapse the mechanically expandable member.

18. The method of claim 16, comprising delivering the ablation device to the treatment site using an endoscope.

19. The method of claim 16, comprising applying energy to the diseased tissue for a sufficient time to ablate the diseased tissue.

20. The method of claim 17, comprising moving the mechanically expandable member to a second treatment site in the collapsed configuration and expanding the mechanically expandable member at the second site by longitudinally moving the first shaft relative to the second shaft.
